(19) United States
(12) Patent Application Publication
(10) Pub. No.: US 2011/0268675 A1
(43) Pub. Date: Nov. 3, 2011

(54) Cosmetic Composition Using Low Molecular Weight Dimethicone

(75) Inventors: Ann Ureneck, Red Bank, NJ (US); Joseph Callelo, Bridgewater, NJ (US); Anjali Patil, Westfield, NJ (US); Julio Russ, Westfield, NJ (US); Christopher Frankfurt, Union Beach, NJ (US)

(73) Assignee: Revlon Consumer Products Corporation, New York, NY (US)

(21) Appl. No.: 13/141,013

(22) PCT Filed: Jan. 26, 2010
(86) PCT No.: PCT/US10/22109

§ 371 Date: Jun. 20, 2011

Related U.S. Application Data
(60) Provisional application No. 61/147,594, filed on Jan. 27, 2009.

(51) Int. Cl. A61K 8/25; A61Q 17/04; A61Q 1/04

(52) U.S. Cl. 424/59; 424/724; 424/64

(57) ABSTRACT

Cosmetic compositions which include low molecular weight non-volatile oils, such as dimethicone, along with silica and a volatile solvent are provided. Such compositions exhibit surprisingly beneficial properties such as long wear, resistance to color transfer, low tack and excellent comfort and shine.

The present invention relates to the field of cosmetic compositions applied to skin.

Cosmetic compositions or cosmetics are generally defined as compositions suitable for application to the human body. Color is one of the most important reasons for wearing cosmetics and color-containing cosmetics must be very carefully formulated to provide maximum wear and effect.

One of the long-standing problems with cosmetics, such as lip products, is the tendency of the cosmetic to blot or transfer from the skin onto other surfaces such as glassware, silverware, or clothing. This not only creates soiling, but forces the cosmetic user to reapply cosmetics at fairly short intervals.

For example, traditional cosmetic compositions are either water and oil emulsions containing pigments, or they can be anhydrous systems containing waxes, oils and pigments. These formulations are applied and blended into the skin to provide color and correct skin topography to provide an even, smooth appearance. The cosmetic is simply deposited on the surface of the skin and if touched with fingers the cosmetic may transfer or become blotchy and uneven. In addition, perspiration or sebum can break through the cosmetic and cause running or smearing. If skin with cosmetic applied comes into contact with clothing, the clothing may become soiled.

Cosmetic compositions containing trimethylated silica partially solve this problem and have been used for various cosmetic and pharmaceutical applications. For example, U.S. Pat. No. 5,800,816 describes cosmetic compositions having improved transfer resistance comprising trimethylated silica, a volatile solvent and a nonvolatile oil. Such compositions have long-lasting adherence to skin and yield a film which is not disturbed when blotted to remove sebum or perspiration. In addition such cosmetics are not readily transferred to clothing or utensils and exhibit reduced permeability to oil and water.

Unfortunately, cosmetic compositions, which use trimethylated silica and other traditional cosmetic and pharmaceutical ingredients, can still exhibit undesirable properties such as being somewhat tacky to the touch and lacking comfort and high shine desirable by many customers. Such compositions, therefore, sometimes require an additional top coat for an optimal look and feel. Accordingly, there still exists a need for cosmetic compositions which not only have long-lasting adherence to skin and resistance to transference to cloths or other objects, but which also are more comfortable, less tacky, and/or have a high shine, in a single step, thereby, eliminating the need for a top coat.

The present invention relates to the surprising discovery that the use of lower molecular weight nonvolatile silicone oils, such as dimethicone, dimethicone copolyol, or mixtures thereof, where such oils have molecular weights of about 60,000 Daltons or less, together with silica, such as trimethylated silica, volatile solvents and other cosmetic ingredients provides surprising results. Such compositions provide lip products that are long-lasting and have excellent transfer resistance, less tack, more comfort, and a high shine.

One object of the present invention is to provide a lip product formulation which has long-lasting adherence to skin and yields a film not disturbed when blotted to remove sebum or perspiration, is not readily transfer to clothing or utensils. Another attribute of the lip product is that it is less tacky to the touch when applied to the lip.

Another object of the invention is to provide cosmetic compositions which are more comfortable when applied to the lip.

Yet another object of the invention is to provide cosmetic compositions with high shine when applied to the lip.

One embodiment of the present invention provides a cosmetic composition comprising silica, a volatile solvent and a non-volatile oil having a molecular weight of about 60,000 Daltons or less.

Another embodiment of the present invention provides a cosmetic composition comprising:
a) from about 0.1-60% by weight of trimethylated silica,
b) from about 0.1-60% by weight of a volatile solvent having a viscosity of 0.5 to 100 centipoise at 25° C., and
c) 0.1-60% of a nonvolatile silicone oil selected from the group consisting of dimethicone, dimethicone copolyol, and mixtures thereof, having a molecular weight of about 60,000 Daltons or less.

Another embodiment of the present invention provides a cosmetic composition comprising:
a) from about 0.1-60% by weight of trimethylated silica,
b) from about 0.1-60% by weight of a volatile solvent having a viscosity of 0.5 to 100 centipoise at 25° C., and
c) 0.1-60% of a nonvolatile oil selected from the group consisting of dimethicone, dimethicone copolyol, and mixtures thereof, having a molecular weight of about 60,000 Daltons or less, and
d) 0.1-80% of a cosmetically acceptable carrier.

### DETAILED DESCRIPTION

The present invention relates to the surprising discovery that the use of lower molecular weight nonvolatile silicone oils, such as dimethicone, dimethicone copolyol, or mixtures thereof, at or less than about 60,000 Daltons, together with silica, such as trimethylated silica and a volatile solvent provides a single step lip product compositions with excellent transfer resistance, less tack, more comfort, and a high shine.

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods of the invention and how to make and use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. The use of examples anywhere in this specification, including examples of any terms discussed herein, is illustrative only, and in no way limits the scope and meaning of the invention or of any exemplified term. Likewise, the invention is not limited to the examples presented.

As used herein, the term “about” or “approximately” generally means within 20 percent, preferably within 10 percent, and more preferably within 5 percent of a given value or range.

As used herein, the term “trimethylated silica” means trimethylated silicon dioxide particles.

As used herein, the term “volatile” means a compound capable of evaporating from the skin or lips in less than an hour. An example of a volatile compound is a hydrocarbon such as isododecane (2,2,4,4,6-pentamethylheprane), isoparaffin, cyclomethicones, and linear methicones.

As used herein, the term “volatile solvent” means a solvent with a high vapor pressure or low boiling point.

As used herein, the term “lip product” refers to any product that can be applied to a human lip.

As used herein, the term “nonvolatile oil” means a hydrophobic liquid cosmetic ingredient with a low vapor pressure and a boiling point above ambient temperature.

As used herein, the term “antioxidant” means any cosmetically acceptable substance which delays, retards or prevents the decay or deterioration of components of the cosmetic composition due to oxidation.

As used herein, the term “cosmetically acceptable carrier” means a carrier for cosmetic use, which carrier delivers the active components to the intended target and which will not cause harm to humans.

The Low Molecular Weight Non-Volatile Oil Component

Non-volatile oils according to the present invention have a molecular weight of about 60,000 Daltons or less. In certain embodiments, non volatile oils have a molecular weight falling within a range of about 10,000 to about 60,000 Daltons. Without being bound by any particular theory, it is believed that use of relatively low molecular weight non-volatile oils in the specified range may contribute to the surprising levels of shine and comfort of cosmetic compositions by improving plasticization in such compositions. Regardless of the mechanism of action, it has been observed that use of non-volatile oils having molecular weights, at or more than about 60,000 Daltons do not provide the same surprising results.

Non-volatile oils may have a viscosity at or less than about 60,000 centipoise at 25° C.

Non-volatile oils suitable in the composition of the invention include, but are not limited to, nonvolatile silicones. Suitable silicones include, but are not limited to, amodimethicone, bisphenylhexamethicone, dimethicone, dimethicone copolyol, dimethiconol, hexadecyl methicone, methicone, phenyl trimethicone, simethicone, dimethylhydrogensiloxane, stearoxy dimethicone, stearoxytrimethylsilane, vinyldimethicone, and mixtures thereof.

In certain embodiments, dimethicone, diethicone dimethicone copolyol and/or dimethiconol alone or in combination are preferred non-volatile oils. In other embodiments, dimethicone is the non-volatile oil used.

The Volatile Solvent Component

Volatile solvents which may be used according to the present invention generally have low viscosity ranging from about 0.5 to 100 centipose.

Volatile solvents suitable in compositions according to the present invention include volatile low viscosity silicone fluids such as cyclic silicones having the formula:

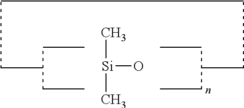

wherein n=1-7. Volatile linear or branched polydimethylsiloxanes are also suitable and generally have from about 2 to 9 silicon atoms and are of the formula:

(CH₃)₃Si—O—[—Si(CH₃)₂—O—]ₙ—Si(CH₃)₃

wherein n=0-7. These silicones include but are not limited to octamethylcyclotetrasiloxane, decamethylcyclopentasiloxane, hexamethyldisiloxane, cyclomethicone, cyclotetrasiloxane, cyclopentasiloxane, and cyclohexasiloxane or mixtures thereof.

Also suitable as the volatile solvent component are straight or branched chain hydrocarbons having 8-20 carbon atoms. In certain embodiments such hydrocarbons preferably have 10-16 carbon atoms. Suitable hydrocarbons are decane, dodecane, tetradecane, tridecane, and C₈₋₂₀ isoparaffins as disclosed in U.S. Pat. Nos. 3,439,088 and 3,818,105, the contents of which are hereby incorporated by reference.

Volatile paraffinic hydrocarbons may have a molecular weight of 160 to 180, a boiling point range of 105 to 320° C., and a viscosity of less than 20 centipoise at 25° C.

Various commercially available C₁₆ isoparaffins, such as isohexadecane, are also suitable. The volatile solvent may be a mixture of volatile silicone and isoparaffins at a ratio of, for example, 1:20 to 20:1. The volatile solvent may range from about 1-40% by weight of the total composition.

The present invention may be used in lip products which are one type of cosmetic composition and the terms may be used interchangeably in this application. Compositions according to the present invention include cosmetics which adhere well to the skin, exhibit reduced transfer resistance, are less tacky, and have a high shine.

The pigments used in the lip product will depend upon the type of formulation. Lip products and similar cosmetics generally contain higher percentages of pigment in the powder phase, usually ranging from about 5-50% of the total cosmetic composition. Generally the pigment: powder ratio ranges from 1:20 to 20:1.

Volatile solvents may comprise a mixture of volatile silicone and a volatile hydrocarbon, and the dry particulate matter may comprise a combination of pigments and powders.

Other Components

Cosmetics and personal care products usually contain conventional excipients which may be used, for example, to modify various properties of the composition and to improve aesthetics. Cosmetic compositions according to the present invention may include excipients such as, for example, emulsifiers, colorants, waxes, oils, preservatives, thickeners, and/or conditioning agents or treatment agents. Commonly used natural and synthetic excipients are described, for example, in International Cosmetic Ingredient Dictionary and Handbook, Twelfth Edition 2008, ISBN-10: 1882621433, (hereinafter “Cosmetic Handbook”) and CTFA ingredient information (http://www.ctfa-online.org/pls/ctfa_online. home), the content of which is hereby incorporated by reference in its entirety for excipients.

Emulsifiers are typically used to help blend ingredients which otherwise would be immiscible. Emulsifiers may be synthetic or natural. Natural emulsifiers may include, but are not limited to, olive oil, olive oil/wheat protein, olive oil/oat protein, sucrose esters, rice bran emulsifiers and/or various other food and pharmaceutical grade emulsifiers, alone or in combination.

Synthetic emulsifiers may include, but are not limited to, silicone emulsifiers, such as dimethicone copolyols; sulfonates and sulfonic acids derivatives; phosphorous organic derivatives; sugar esters; fatty esters, such as sorbitan monolaurate, sorbitan stearate, sorbitan laurate, sorbitan palmitate, sorbitan oleate; polyesters/PEG (polyethylene glycol) derivatives, such as Polysorbate 20 (polyethylene glycol 20 sorbitan monolaurate); fatty acid esters of fatty alcohols, such as glyceryl stearate, isopropyl stearate, hexyl laurate; fatty acid amides; acyl lactylates; alkoxylated compounds, such as alkoxylated block polymers, alcohols, alkylphenols, amines, amides, fatty esters, fatty acids, oils, sugar esters and polyesters, fatty acid esters of fatty alcohols, and ethers of fatty alcohols; carboxylated alcohol ethoxylates and alkylphenol ethoxylates; carboxylic acides/fatty acids, and mixtures thereof.

Pigments are typically used to provide relatively uniform color to the final cosmetic composition. Pigments may be synthetic or natural. Natural pigments may include pigments or plant-derived colors. Natural pigments may be inorganic (mineral) or organic, white or non-white, and coated or uncoated particles. Natural pigments may include, for example, cerium oxide, iron oxide, titanium dioxide, zinc oxide, zirconium oxide, carbon black, manganese violet, ultramarine blue, D&C and FD&C colors, azo, indigoid, insoluble metallic salts of certified color additives, referred to as the Lakes, and the like, and mixtures thereof.

Compositions according to the present invention may include waxes. Suitable waxes may have a melting point ranging from 35 to 120° C., and include, for example, natural and synthetic waxes. Natural waxes may include, for example, bayberry wax, beeswax, candelilla wax, carnauba wax, hydrogenated jojoba oil, hydrogenated jojoba wax, hydrogenated microcrystalline wax, hydrogenated rice bran wax, Japan wax, jojoba butter, jojoba oil, jojoba wax, mink, ouricury, ozokerite, rice bran, and/or shellac. Synthetic waxes may include, for example, ceresin, cetyl esters, lanolin wax, microcrystalline wax, montan, montan acid wax, paraffin, PEG-6 beeswax, PEG-8 beeswax, polyolefin, sulfurized jojoba oil, synthetic beeswax, synthetic candelilla wax, synthetic carnauba wax, synthetic Japan wax, synthetic jojoba oil, synthetic wax, stearoxy dimethicone, dimethicone behenate, stearyl dimethicone, and synthetic homo- and copolymer waxes from the ethylene series or mixtures thereof, waxes obtained by Fischer-Tropsch synthesis, waxy copolymers and esters thereof, and silicone waxes.

Compositions according to the present invention also may include one or more essential and natural oils. Essential oils may be synthetic or natural. Natural essential oils may include bergamot, chamomile german, chamomile maroc, chamomile roman, cinnamon zeylanicum, clove buds, eucalyptus globulus, frankincense, fennel, hyssop, juniper, lemon grass, mountain savory, niaouli, red thyme, rosemary, rose geranium, tagestes, and ylang ylang. Natural oils may include, for example, jojoba oil, sweet almond oil, coconut oil, shea butter, mango butter, and/or aloe vera butter or mixtures thereof.

Synthetic essential oils may include, for example, esters, such as acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, PEG-4 diheptanoate, hydrogenated castor oil, isotridecyl isononanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, tridecyl octanoate, octyl palmitate, dioctyl malate, tridecyl octanoate, myristyl myristate, octododecanol; fatty alcohols such as oleyl alcohol, isocetyl alcohol; and also silicone oils, isoparaffins, hydrogenated polyisobutene, petrolatum, lanolin derivatives, and sorbitan derivatives. Other natural and synthetic oils may be found in the Cosmetic Handbook and CTFA ingredient information.

Compositions according to the present invention also may include preservatives. Preservatives may be either synthetic or natural and may be used to inhibit growth of undesirable microorganisms. Natural preservatives may include black currant fruit extract, aspen bark, radish root, and sorbic acid, alone or in combination.

Synthetic preservatives may include, for example, methylparaben, ethylparaben, propylparaben, imidazolidinyl urea, diazolidinyl urea, DMDM hydantoin, isothiazolinones, chlorinated aromatic compounds, para-hydroxybenzoic acids/parabens, alone or in combination. Other natural and synthetic preservatives may be found in the Cosmetic Handbook and CTFA ingredient information.

Compositions according to the present invention also may include thickeners. Thickeners may be either synthetic or natural. Thickeners may be used to gel or thicken cosmetic compositions to provide, for example, better deposition properties. Natural thickeners may include waxes, gums and powders and mixtures thereof. Natural waxes may include beeswax, carnauba, and/or candelilla and mixtures thereof. Natural gums may include acacia, xanthan, schelortium (amigel), and/or cellulose and mixtures thereof. Natural powders may include clay, diatomaceous earth, fuller's earth, silica, silica shells or spherical silica, fumed silica, spherical silica, hydrated silica, silica silylate, mica, titanated mica, talc, cellulose or spherical cellulose beads, microcrystalline cellulose, corn starch, rice starch, glyceryl starch, soy flour, walnut shell powder, agar, sericite, dextran, nylon, silk powder, chalk, calcium carbonate, bismuth oxychloride, iron oxide, titanium dioxide, aluminum silicate, magnesium aluminum silicate, calcium silicate, magnesium trisilicate, aluminum starch octenylsuccinate, bentonite, hectorite, kaolin, maltodextrin, montmorillonite, zinc laurate, zinc myristate, zinc rosinate, alumina, attapulgite, tin oxide, titanium hydroxide, trimagnesium phosphate, or mixtures thereof.

Synthetic thickeners may include, for example, AMP isostearoyl hydrolyzed collagen, AMP isostearoyl hydrolyzed wheat protein, cetyl hydroxyethylcellulose, chondroitin sulfate, cocoamidopropyldimethylamine C₈₋₁₆ isoalkysuccinyl lactoglobulin sulfonate, cocodimonium hydroxypropyl hydrolyzed collagen, distarch phosphate, ethyl ester of hydrolyzed animal protein, guar hydroxypropyltrimonium chloride, hydrolyzed animal or plant protein, hydroxypropyl guar, hydroxypropylmethyl cellulose, hydroxypropyl cellulose, isostearoyl hydrolyzed collagen, methylcellulose, nitrocellulose, nonoxynyl hydroxyethylcellulose, acrylate polymers, acrylamine polymers, acrylic acid polymers (carbomer), PVMIMA Decadiene crosspolymers, polyvinylpyrrolidone polymers, silicone oils, polyethylene thickeners, aluminum starch octenyl succinate, trihydroxystearin, and mixtures thereof. Other natural and synthetic thickeners may be found in the Cosmetic Handbook and CTFA ingredient inform.

Compositions according to the present invention also may be incorporated into water and oil emulsion cosmetic compositions. Cosmetic generally contains water, and pigment in addition to an oil phase. Suitable cosmetic compositions may comprise:
0.1-20% trimethylated silica
0.1-40% of a volatile solvent having a viscosity of 0.5 to 350 centistokes,
0.1-25% of a nonvolatile nonfluorinated silicone oil having a molecular weight of about 60,000 Daltons or less,
0.1-70% dry particulate matter having a particle size of 0.02 to 100 microns, and
0.1-50% water.

Non-volatile oils in such cosmetic compositions may include dimethicone, dimethicone copolyol, or a mixture thereof, and the pigment to powder ratio may be 1:20 to 20:1.

Cosmetically acceptable vehicles also include lip products. Lip products generally include wax, oil, and pigment. Lip product compositions according to the present invention may comprise:
0.1-60% trimethylated silica,
0.1-60% volatile solvent,
0.1-60% nonvolatile oil having a molecular weight of about 60,000 Daltons or less,
0.1-80% cosmetically acceptable carrier,
0.1-40% wax.

In lip product compositions, non-volatile oils may include dimethicone, dimethicone copolyol, or a mixture thereof. Lip product compositions also may contain one or more waxes, film formers, sunscreens, preservatives, antioxidants, emulsifiers and thickeners, many examples of which are included in the personal Care Products Council website (http://online.personalcare council.org or as updated) and said examples are incorporated by reference.

Examples of sunscreen include but are not limited to the following. In particular, about 0.01-10% by weight of various sunscreen compounds such as PABA and derivatives thereof can be incorporated into a cream or lotion according to embodiments of the present invention.

The dry particulate matter may include titanium dioxide and other powdered materials which provide sunscreen protection.

The invention will be further described in connection with the following illustrative examples.

### EXAMPLES

### Example 1

As shown in Table 1, low molecular weight dimethicone was incorporated into a lip product formulation.

TABLE 1

| | Wet percentage |
|---|---|
| CTFA Name | (%) |
| Isododecane | 43.0 |
| Trimethylsiloxysilicate | 20.0 |
| Dimethicone | 15.9 |
| Disteardimonium | 5.0 |
| hectorite | |
| C12-15 alkyl benzoate | 1.0 |
| Polyethylene | 3.0 |
| BHT | 0.1 |
| Sorbic Acid | 0.2 |
| Silk powder | 0.1 |
| Yellow 5 lake | 0.3 |
| Red 7 lake | 0.3 |
| Red 6 lake | 0.1 |
| Titanium dioxide | 1.0 |
| Mica | 10.0 |
| Total | 100.00 |

Lip products described by the formulation in Table 2 have a nice feel and less tack than compositions using higher molecular weight non-volatile oils. Such compositions also allow longer wear time.

### Example 2

As shown in Table 3 below, lip product formulations incorporating dimethicone at molecular weights ranging from about 10,000 to 600,000 Daltons were compared.

TABLE 2

| CTFA Name | % w/w | % w/w | % w/w | % w/w | % w/w |
|---|---|---|---|---|---|
| Isododecane | 54.47 | 54.47 | 54.47 | 54.47 | 54.47 |
| Trimethyl/Siloxy Silicate | 26.45 | 26.45 | — | 26.45 | 26.45 |
| Dimethicone 600,000 | 19.08 | — | — | — | — |
| MW | | | | | |
| Dimethicone 60,000 MW | — | 19.08 | 19.08 | — | — |
| Dimethicone 10,000 MW | — | — | — | 19.08 | — |
| Dimethicone | — | — | — | — | 19.08 |
| 300,000 MW | | | | | |
| Gloss (1 hr, 40° C.) | | | | | |
| 3 mil | | | | | |
| Black | 23.3 | 71.6 | | 75.5 | 75.5 |
| White | 24.6 | 66.6 | | 76.3 | 76.6 |

### Gloss Method

Drawdown solution onto 10¼″×7½″ LENETA FORM 5C drawdown card (black/white) using 3 mil drawdown bar
Allow to dry in 40° C. chamber for 1 hour
Measure gloss at 60° over both black and white portion of the card
Take 3 readings each and average the values

It was determined that use of dimethicone having molecular weights of about 10,000 or 60,000 Daltons results in lip products which have surprising comfort and shine when compared to compositions which use dimethicone which have higher molecular weights (e.g. 300,000 and 600,000).

### Example 3

Comfort of the lip product was tested on a DermaLab USB Skin Analysis System (Cortex Technology, Denmark) using methods as described in The Handbook of Non-Invasive Methods and the Skin, Second Edition, Editors: Jorgen Seurp, Gregor B. E. Jemec, Gary L. Cove, 2006 CRC Press (Taylor & Francis Group, LLC), methods hereby incorporated by reference. As shown in Table 4, lip product compositions according to Example 1 were tested using dimethicone at various molecular weights and a sample size of 15. This Example shows that lip product compositions with lower molecular weight dimethicone have higher flexibility.

TABLE 4

| Viscosity of Dimethicone | Result |
|---|---|
| 60K Pre (kPa) | 50.793 ± 4.625 |
| 60K-Post (kPa) | 42.753 ± 7.087 |
| 300K-Pre (kPa) | 40.960 ± 9.585 |
| 300K-Post (kPa) | 36.787 ± 8.008 |
| 600K-Pre (kPa) | 45.660 ± 8.324 |
| 600K-Post (kPa) | 41.253 ± 8.313 |

What is claimed is:

1. A lip product composition comprising silica, a volatile solvent and a non-volatile oil having a molecular weight of about 60,000 Daltons or less.

2. The composition of claim 1, wherein the non-volatile oil is selected from the group consisting of dimethicone, dimethicone copolyol, and mixtures thereof.

3. The composition of claim 1, wherein the non-volatile oil has a molecular weight at a range between about 10,000 to about 60,000 Daltons.

4. The composition of claim 1, wherein the volatile solvent has as viscosity of 0.5 to 100 centipoise at 25° C.

5. The composition of claim 1, wherein the non-volatile oil comprises dimethicone.

6. The composition of claim 1, further comprising a cosmetically acceptable carrier.

7. The composition of claim 6, wherein the acceptable carrier comprises a film former, a wax, an emulsifier, a pigment, a preservative, an essential oil, a natural oil, an antioxidant, a thickener, a sunscreen, and mixtures thereof.

8. The composition of claim 1, wherein the cosmetic composition is a lip product.

9. A cosmetic composition comprising:
a) from about 0.1-60% by weight of trimethylated silica,
b) from about 0.1-60% by weight of a volatile solvent having a viscosity of 0.5 to 100 centipoise at 25° C., and
c) 0.1-60% of a non-volatile, silicone oil selected from the group consisting of dimethicone, dimethicone copolyol, and mixtures thereof, having a molecular weight of about 60,000 Daltons or less.

10. The composition of claim 9, wherein the non-volatile, silicone oil comprises dimethicone.

11. The composition of claim 9, wherein the non-volatile, silicone oil has a molecular weight at a range between about 10,000 to about 60,000 Daltons.

12. The composition of claim 9, further comprising a cosmetically acceptable carrier, wherein said carrier comprises from about 0.1-80% of the composition by weight.

13. The composition of claim 11, wherein the cosmetic composition is lip product.

14. A cosmetic composition comprising a volatile solvent and a non-volatile silicone oil having a molecular weight of about 60,000 Daltons or less, wherein said cosmetic composition exhibits less tack and greater shine than a comparable composition comprising a non-volatile oil having a molecular weight substantially higher than 60,000 Daltons.

15. The cosmetic composition of claim 14 comprising:
a) from about 0.1-60% by weight of trimethylated silica,
b) from about 0.1-60% by weight of a volatile solvent having a viscosity of 0.5 to 100 centipoise at 25° C., and
c) 0.1-60% of a non-volatile, non-fluorinated silicone oil selected from the group consisting of dimethicone, dimethicone copolyol, and mixtures thereof, having a molecular weight about 60,000 Daltons or less.

16. The composition of claim 15, wherein the non-volatile, non-fluorinated silicone oil comprises dimethicone.

17. The composition of claim 15, wherein the non-volatile, non-fluorinated silicone oil has a molecular weight at a range between about 10,000 to about 60,000 Daltons.

18. The composition of claim 15, further comprising a cosmetically acceptable carrier, wherein said carrier comprises from about 0.1-80% of the composition by weight.

19. The composition of claim 18, wherein the cosmetic composition is lip product.

20. The composition of claim 19, further comprising at least one pigment.
